(19) United States
(12) Patent Application Publication
(10) Pub. No.: US 2023/0173513 A1
(43) Pub. Date: Jun. 8, 2023

(54) AEROSOLISING DEVICE

(71) Applicant: PIETER VAN WEENEN & CO. GmbH, The House of Innovation, Waldkirch (DE)

(72) Inventors: Karsten Schwörer, Wyhl (DE); Patrick Weindl, St. Peter (DE); Oliver Wisser, Waldkirch (DE)

(21) Appl. No.: 17/799,477

(22) PCT Filed: Apr. 12, 2021
(86) PCT No.: PCT/EP2021/059466

§ 371 Date: Jan. 30, 2023

(30) Foreign Application Priority Data
Feb. 12, 2020 (DE) 10 2020 000 903.4

(51) Int. Cl. B05B 7/06; B05B 7/10; C12M 1/00

(52) U.S. Cl. CPC B05B 7/066; B05B 7/10; C12M 29/08

(57) ABSTRACT

An aerosolization device for an aerosol wetting chamber comprising a receiving opening for receiving the aerosolization device and adapted for wetting samples disposed in the aerosol wetting chamber with aerosol, the aerosolization device comprising a main body, an aerosol deflector and a swirling device.

### FIELD OF THE INVENTION

The present invention relates generally to devices for exposing biological samples to aerosol and, more particularly, to devices for supplying aerosol to a space in which biological samples are to be exposed to aerosol.

### BACKGROUND

The investigation of the effects of substances (e.g. active substances, toxins, nanoparticles, fibres or dusts in solution or suspension) on biological material (e.g. cell cultures, tissues, bacteria, skin cells, organic secretions) is of increasing interest, for example in order to be able to make predictions about the usefulness or harmfulness of a substance in terms of the biological reaction or its kinetics, i.e. the speed of a reaction of an organism to the substance in question.

One approach is to obtain an aerosol from a substance to be investigated and to expose samples to it. Arrangements with a substantially enclosed space with aerosol can be used for this purpose. Basically, such aerosol wetting arrangements comprise a base at/in which biological samples are arranged (for example by means of so-called transwell inserts), an aerosol wetting chamber and an aerosolization device. An example of such an arrangement is described in DE 10 2013 005 010 A1. Furthermore, DE 101 04 012 A1 discloses an aerosolization device comprising a main body and a downstream aerosol deflector, wherein the aerosol deflector performs the function of a baffle for additional aerosolization.

### OBJECT OF THE INVENTION

The object of the invention is to provide solutions for more flexible aerosolization devices which are improved with regard to the exposure of the sample to an aerosol.

### BRIEF DESCRIPTION OF THE INVENTION

To solve this object, the present invention provides subject matters according to the appended claims.

Thus, an aerosolization device is provided for use with an aerosol wetting chamber having a receiving opening for receiving the aerosolization device and adapted for wetting samples disposed in the aerosol wetting chamber with aerosol.

The aerosolization device may comprise:
a main body with an aerosol passageway, which includes an outlet for discharging aerosol in a discharge direction,
an aerosol deflector disposed downstream of the outlet in the discharge direction and having at least one deflecting surface extending transverse to the discharge direction and adapted to deflect aerosol emerging from the outlet in a direction different from the discharge direction,
a swirling device arranged downstream of the aerosol deflector in the discharge direction and comprising at least one movable element adapted to set surrounding fluid in motion.

The at least one deflecting surface may be configured to match the geometry of an aerosol wetting chamber to provide a desired distribution of aerosol there.

Further additional or alternative parameters in the design of the at least one deflecting surface are properties of the aerosol (e.g. density, specific weight, velocity, particle size, temperature, etc.), the temperature inside the aerosol wetting chamber, the arrangement of samples in the aerosol wetting chamber etc.

Variants are provided in which the deflecting surface has the shape of the lateral surface of a cone, more specifically a conical shape, with the lateral surface comprising areas with respect to the discharge direction—which are not constant as in a cone—the angles of which differ at least once with respect to the discharge direction. For example, the areas become “more obtuse” and/or “flatter” once or several times, e.g. in order to optimize the flow.

The at least one deflecting surface may comprise at least three corners.

There may be at least two deflecting surfaces, each extending transverse to the discharge direction, wherein an angle between the discharge direction and the respective plane of the deflecting surface may be the same or different.

The at least one deflecting surface may have the shape of a lateral surface of a cone with a first angle between the cone axis and the surface line of the cone, which is adjoined by the lateral surface of a truncated cone with a second angle between the cone axis and the surface line of the truncated cone.

This way, the at least one deflecting surface can have two areas. For example, the first area may begin at the top at an upper end of the deflecting surface and extend to a transition area where the second area may begin and extend to a lower end of the deflecting surface.

The first area may have the shape of a lateral surface of a cone, with a first half cone angle. A half cone angle is understood here to be the angle between the cone axis and the surface line.

The second area may have the shape of a lateral surface of a truncated cone, with a second half cone angle.

The first cone angle may be smaller than the second cone angle. For example, the first cone angle may be in the range between 30° and 60° (e.g. about 45°) and the second cone angle may be in the range between 40° and 80° (e.g. about 60°).

The first cone angle may be greater than the second cone angle; in such variants, it can be said that from the perspective of aerosol coming from above (e.g. from the aerosol receiving device 20), the deflecting surface 62 is first flatter and then steeper.

For example, the first cone angle may be in the range between 40° and 80° (e.g. about 60°) and the second cone angle may be in the range between 30° and 60° (e.g. about 45°).

In further variants, the at least one deflecting surface may be curved, whereby the curvature may be concave or convex.

In further variants, the at least one deflecting surface may have at least two areas that have a different curvature. For example, the first area may begin at the top at an upper end of the deflecting surface and extend to a transition point where the second area may begin and extend to a lower end of the deflecting surface. The first and second area may have different curvature, for example, the curvature of the first area is greater than that of the second area, or vice versa. The curvatures can be concave and/or convex.

The at least one deflecting surface may have the shape of a lateral surface of at least one of the following:
a cone or truncated cone, or
a sphere or hemisphere, or
a segment of a sphere or hemisphere, or
a cylinder or segment of a cylinder, or
a prism or prism segment, or
a pyramid or truncated pyramid.

The at least one deflecting surface can be formed on a supporting body.

The supporting body may have a seat for at least partially receiving the swirling device.

A drive of the swirling device may be disposed in the seat of the supporting body.

The movable element of the swirling device may comprise at least one vane (wing, rotor blade) that is rotatable about a rotation axis.

The rotation axis may extend in a direction parallel or perpendicular at an angle to the discharge direction.

The movable element of the swirling device may be disposed in a space that comprises at least one opening.

The supporting body may comprise the space in which the movable element of the swirling device is disposed.

The main body may comprise at least one fluid supply passageway comprising an inlet for supplying fluid to be supplied to the aerosol wetting chamber, and an outlet for discharging the fluid to be supplied to the aerosol wetting chamber into the aerosol wetting chamber.

The main body may comprise at least one fluid discharge passageway comprising an inlet for supplying fluid to be discharged from the aerosol wetting chamber, and an outlet for discharging fluid to be discharged from the aerosol wetting chamber into the aerosol wetting chamber.

The main body may comprise at least one cable feed-through, adapted to feed through a line for controlling and/or supplying energy to the swirling device. The line feed-through may be configured to provide an electrical insulation against aerosol.

The aerosol deflector and the main body may be connected by an elongate connecting member extending in the discharge direction.

The at least one connecting member may be releasably connected with at least one of the aerosol deflector and the main body.

The at least one connecting member may comprise a passageway extending in the longitudinal direction of the connecting member.

The passageway of the connecting member may be adapted to feed through a line for controlling and/or supplying energy to the swirling device. The passageway of the connecting member may be configured to provide an electrical insulation against aerosol.

The main body may comprise at least one sealing element at one outer surface, which is adapted to provide sealing against the receiving opening of the aerosol wetting chamber when the aerosolization device is received in the receiving opening of the aerosol wetting chamber.

The aerosol passageway may extend in the discharge direction.

The aerosol passageway may comprise at least one sealing element at an inner surface.

The aerosolization device may comprise a port for an aerosol feed line for feeding aerosol of an external source, the port being arranged upstream of the aerosol passageway and being in fluid communication with the aerosol passageway.

The aerosolization device may comprise an aerosol receiving device with an aerosol generation chamber comprising an inlet for loading a substance for producing an aerosol into the aerosol generating chamber, and an outlet for feeding aerosol produced from the substance from the aerosol generating chamber to the aerosol passageway.

The aerosol receiving device may comprise a connecting part extending from the aerosol generation chamber which is configured to provide a connection with the aerosol passageway.

The connecting part of the aerosol receiving device may be configured to be at least partly introduced into an inlet of the aerosol passageway.

The connecting part of the aerosol receiving device may comprise at least one sealing element at an outer surface.

At least one membrane may be arranged in the aerosol generation chamber which is adapted to allow aerosol present in the aerosol generation chamber in an area between the inlet of the aerosol generation chamber and the membrane to pass through in the direction toward the outlet of the aerosol generation chamber.

The at least one membrane may comprise a controllable oscillating membrane.

### BRIEF DESCRIPTION OF THE DRAWINGS

Below, variants of the present invention are described with reference to the drawings.

FIG. 1 shows a schematic illustration of an aerosol wetting arrangement.

FIG. 2 shows a schematic illustration of an aerosol receiving device usable as an aerosolization device.

FIG. 3A-4B show schematic illustrations of a variant of an aerosolization device.

FIG. 4A-4B show schematic illustrations of a variant of an aerosolization device with fluid supply and removal.

FIGS. 5A-5E show schematic illustrations of a variant of an aerosolization device with an aerosol deflector and swirling device.

FIG. 6A-4C show schematic illustrations of a variant of an aerosolization device without aerosol receiving device.

The explanations given with respect to a drawing or variant generally apply to all other drawings and variants accordingly, unless otherwise stated. Furthermore, reference numerals introduced in previous drawings will not be repeated in all subsequent drawings; nonetheless, reference numerals not shown therein shall be deemed to be disclosed therein as well.

### DETAILED DESCRIPTION

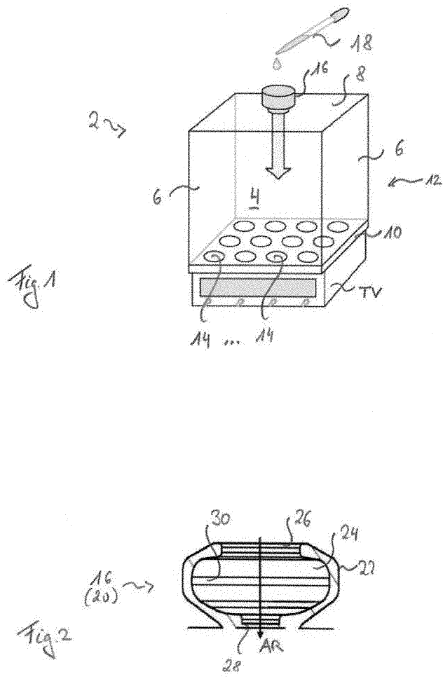

As stated above, aerosol wetting arrangements comprise a base at/in which samples are arranged (for example by means of so-called transwell inserts), an aerosol wetting chamber and an aerosolization device. FIG. 1 illustrates in a schematically simplified manner an exemplary aerosol wetting arrangement 2.

The aerosol wetting chamber 4 of the aerosol wetting arrangement 2 is limited by side walls 6 (of which the one on the front and the one on the right have been provided with numerals) and a top 8 as well as the base 10. The side walls 6 and top 8 may together be referred to as hood 12. The hood 12 is generally releasably connected with a base 10 in order to insert and remove samples in corresponding wells 14, but also in order to be able to clean the hood 12 easily. The top 8 of the well 12 can be releasably connected with the side walls 6. A temperature regulator TV may be coupled to the base 10 in order to control and set the temperature of samples and/or their environment.

The hood 12 comprises an opening at which an aerosolization device 16 is arranged.

The aerosolization device 16 is a device to which a substance from which an aerosol is to be generated is supplied, and which gives off the generated aerosol into the aerosol wetting chamber. Here, an aerosolization device is understood to be the device which as such is configured to supply aerosol to an aerosol wetting chamber.

Substances may be supplied to an aerosolization device e.g. by means of pipetting 18, as shown in FIG. 1. The aerosolization device comprises means to generate an aerosol from the supplied substance. An oscillating membrane can be used to this end.

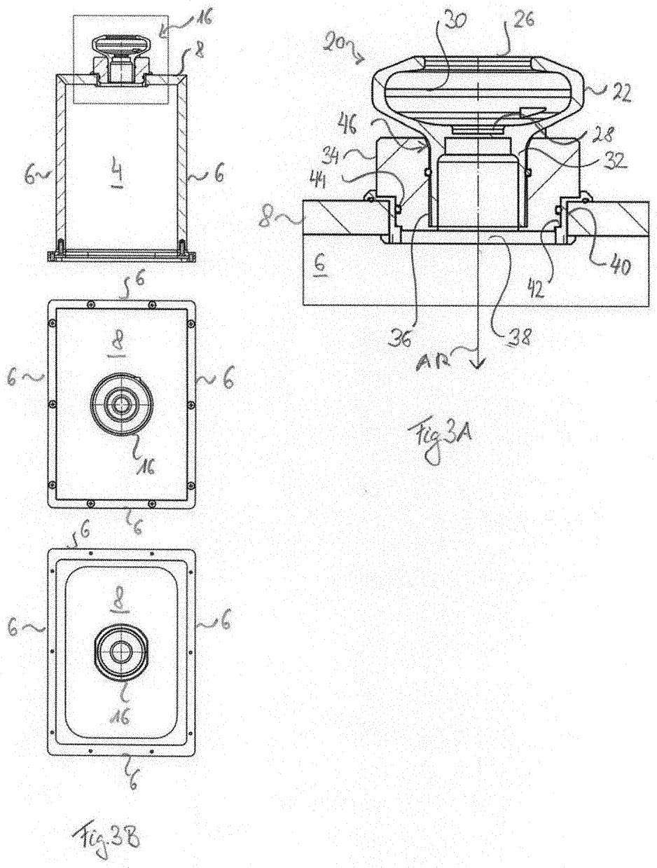

FIG. 2 illustrates in a schematically simplified manner an exemplary device which may serve as an independent aerosolization device, i.e. which is able to supply aerosol to an aerosol wetting chamber substantially without any further components, or which may form part of an aerosolization device, as explained below. In order to take the latter case into account, the device according to FIG. 2 will also be referred to as aerosol receiving device in order to distinguish the term from an aerosolization device of which it forms a part.

The aerosol receiving device 20 of FIG. 2 comprises a housing 22 that defines an aerosol generation chamber 24 and an inlet 26 for loading a substance for producing an aerosol into the aerosol generating chamber 24, and an outlet 28 for discharging aerosol produced from the substance from the aerosol generating chamber.

A membrane 30 is disposed in the aerosol generation chamber 24 which may be used to generate an aerosol from a substance present above the membrane 30 in the aerosol generation chamber 24. For example, an oscillating membrane can be used which is electrically excited to generate an aerosol which may exit the aerosol generation chamber 24 through the outlet 28 at the bottom.

When the outlet 28 of the device of FIG. 2, as shown in FIG. 1, is brought in fluid communication with an inlet of the aerosol wetting chamber 4, aerosol may enter into the aerosol wetting chamber 4.

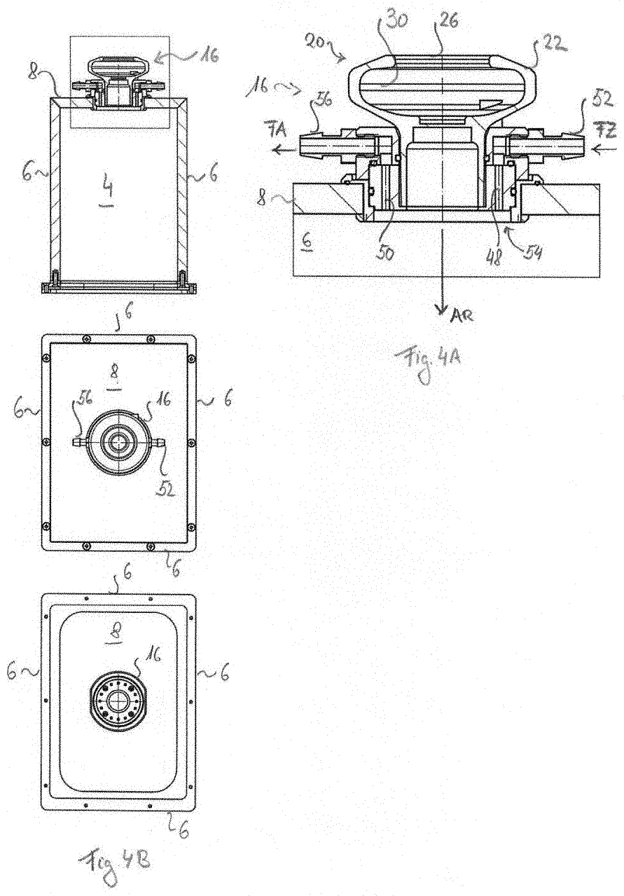

FIGS. 3A and 3B show a variant in which the device according to FIG. 2 acts as aerosol receiving device 20 (which, in the interaction with further components described hereinafter is “merely” a part of an aerosolization device, and does not, as shown in FIG. 2, act as an independent aerosolization device). The comments above regarding the previous drawings shall apply to the aerosol receiving device, accordingly, unless otherwise stated, and will not be repeated.

The aerosol receiving device 20 comprises an aerosol wetting chamber 4 to which aerosol can be supplied. The aerosol receiving device 20 comprises a housing 22 that defines an aerosol generation chamber 24, an inlet 26 for loading a substance for producing an aerosol into the aerosol generating chamber 24, and an outlet 28 for discharging aerosol produced from the substance from the aerosol generating chamber 24, and a membrane 30 for generating aerosol. The descriptions given above of the aerosol receiving device of FIG. 2 shall apply here accordingly, and will therefore not be repeated.

The housing of FIGS. 3A and 3B further comprises a connecting part 32 which extends downward in the drawing. The connecting part 32 defines a fluid connection from the outlet of the aerosol receiving device 20 out of the latter; therefore, the connecting part 32 may also be referred to as outlet tube.

Furthermore, the aerosolization device 16 comprises a main body 34 with an aerosol passageway 36, which includes an outlet 38 for discharging aerosol in a discharge direction AR.

The main body 34 is configured at what is the bottom side in the drawing for being inserted into a receiving opening 40 of a hood 12 of an aerosol wetting chamber 4. This can be achieved e.g. with a circumferential step 42 of the housing, or any other recess 42 the outer circumference of which substantially corresponds to the inner side of the receiving opening 40.

For a fluid-tight arrangement against the hood 12, the main body 34 may comprise at least one sealing element 44 e.g. in the area of the step/recess 42.

The aerosol receiving device is inserted with its connecting part 32 into the aerosol passageway 36 of the main body 34, e.g. such that the aerosol generation chamber 24, as seen in the drawing, extends above an inlet 46.

For a fluid-tight arrangement between the main body 34 and the aerosol receiving device 20, the outside of the connecting part 32 and/or the inside of the aerosol passage 36 may comprise at least one sealing element (not labelled), for example one or several O-rings.

As schematically shown in FIG. 3B, the aerosol wetting chamber 4 of the aerosol wetting arrangement 2 is limited by side walls 6 and a top 8 which form the hood 12. A base 10 with sample wells 14 (see FIG. 1), which limits the aerosol wetting chamber 4 opposite the top 8, is not shown.

By means of a membrane in the aerosol generation chamber 24, aerosol is produced from a substance present above the membrane 30 in the aerosol generation chamber 24, which may exit the aerosolization device 16 through the outlet 28, the connecting part 32, the aerosol passageway 36 and its outlet 38 in the discharge direction AR, and may be fed into the aerosol wetting chamber 4.

The variant according to FIGS. 3A and 3B allows to provide the aerosol wetting chamber 4 and the aerosolization device 16 as separate components. This may facilitate their respective cleaning. Furthermore, this way, it is possible to use different aerosolization devices in an aerosol wetting chamber (e.g. aerosolization devices with different capacities, different membranes, different aerosol generation means, etc.)

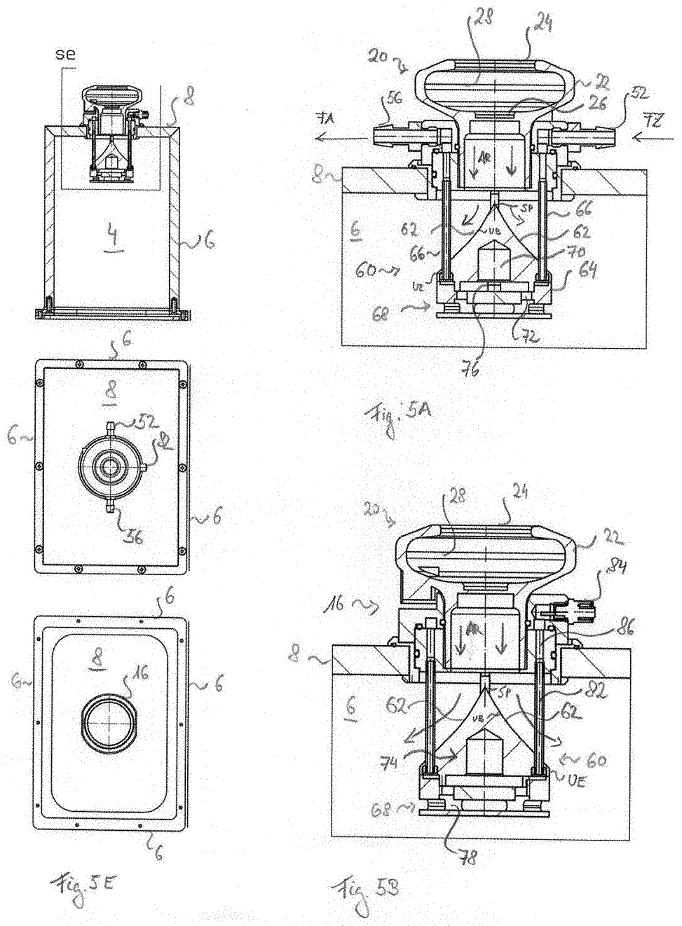

The variant of FIGS. 4A and 4B comprises the components of the variant of FIGS. 3A and 3B. Therefore, the comments above regarding the previous drawings shall apply here accordingly, unless otherwise stated, and will not be repeated.

Here, the main body 34 comprises a fluid supply passageway 48 and a fluid discharge passageway 50. The fluid supply passageway 48 extends from a port 52 through the main body 34 to its bottom side 54. The fluid discharge passageway 50 extends from the bottom side 54 of the main body 34 through the same to a port 56.

The port 52 may e.g. be connected with a source that provides pressurized fluid. For example, oxygen, clean air, protective gas, carbon monoxide or dioxide or other gases may serve as fluid. The source may e.g. be a pump, a compressed gas cylinder, a filter, a pressure reducer or metering devices.

Fluid provided by the source may—as indicated by the arrows FZ—be supplied via the port 52 through a fluid supply passageway 48 through the main body 34 of the aerosol wetting chamber 4. By supplying fluid into the aerosol wetting chamber 4 in this manner, any fluid present in the aerosol wetting chamber 4 may be transported through the fluid discharge passageway 50 through the main body 34 to the port 56—as indicated by the arrows FA. Any fluid discharged in this manner may be collected at the port 56, e.g. by means of a container.

A sink may also be connected to the port 56. The sink may e.g. comprise a pump, filters, or other cleaning devices.

The sink may be used to support the removal of fluid from the aerosol wetting chamber 4. The sink may support the source in supplying fluid, e.g. negative pressure provided by the sink, in conjunction with positive pressure supplied by the source may provide, at least support or replace a fluid supply, i.e. so that no source, but just a sink is employed.

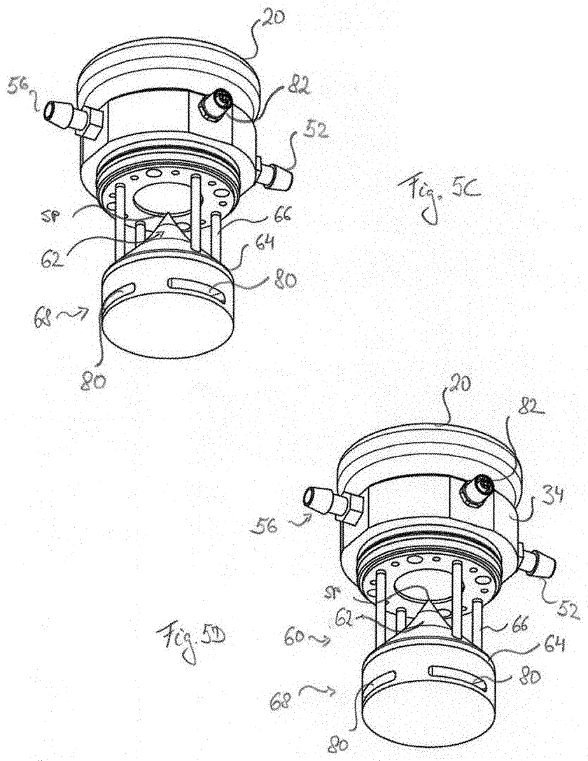

The variant of FIG. 5A to 5E comprises the components of the variant of FIGS. 4A and 4B. Therefore, the comments above regarding the previous drawings shall apply here accordingly, unless otherwise stated, and will not be repeated.

The variant of FIG. 5A to 5E comprises an aerosol deflector 60 arranged downstream of the outlet 38 of the aerosol passageway 36 in the discharge direction AR. The aerosol deflector 60 comprises at least one deflecting surface extending at an angle to the discharge direction AR, which serves to deflect aerosol emerging from the outlet 38 in one or several directions different from the discharge direction AR. The latter is indicated in FIG. 5A to 5E by the arrows AA.

Aerosol coming from the outlet 38 travels substantially in the discharge direction AR into the aerosol wetting chamber 4. This may lead to an uneven distribution in the aerosol wetting chamber 4. For example, in areas of the aerosol wetting chamber 4 below the outlet and adjacent thereto in the discharge direction AR, there may be a higher proportion or concentration of the aerosol than in areas of the aerosol wetting chamber 4 which are closer to the side walls. As a result, samples may be unevenly exposed to aerosol.

The aerosol deflector 60 allows aerosol coming from the outlet 38 to be more evenly distributed in the aerosol wetting chamber 4. The aerosol which initially diffuses in the discharge direction hits upon a deflecting surface 62 which deflects aerosol away from the discharge direction and into areas of the aerosol wetting chamber 4. It is in particular provided to cause the deflection of the aerosol in what is an oblique downward direction in the drawing. Depending on the type of aerosol and/or its discharge rate from the aerosol passageway 36, the aerosol may travel downward along the deflecting surface 62 and/or bounce off it.

In the variant of the aerosol deflector 60, the deflecting surface 62 comprises two areas. In the illustration, the first area begins at the tip SP and reaches down to a transition area UB where the second area begins and extends to the lower end UE of the deflecting surface 62.

According to the drawings, the first area has the shape of a lateral surface of a cone, with a first half cone angle. A half cone angle is understood here to be the angle between the cone axis and the surface line. Furthermore, the second area has the shape of the lateral surface of a truncated cone, with a second half cone angle.

According to the variants shown, the first cone angle is smaller than the second cone angle; in such variants, it can be said that from the perspective of aerosol coming from above (e.g. from the aerosol receiving device 20), the deflecting surface 62 is first steeper and then flatter.

For example, the first cone angle may be in the range between 30° and 60° (e.g. about 45°) and the second cone angle may be in the range between 40° and 80° (e.g. about 60°).

In further variants, the first cone angle may be greater than the second cone angle; in such variants, it can be said that from the perspective of aerosol coming from above (e.g. from the aerosol receiving device 20), the deflecting surface 62 is first flatter and then steeper.

For example, the first cone angle may be in the range between 40° and 80° (e.g. about 60°) and the second cone angle may be in the range between 30° and 60° (e.g. about 45°).

As has already been stated above, in further variants, deflecting surfaces may have the shape of a lateral surface of a truncated cone, a sphere or hemisphere, a segment of a sphere or hemisphere, a cylinder or segment of a cylinder, a prism or prism segment, a pyramid or truncated pyramid.

Instead of one deflecting surface, two or more deflecting surfaces may be used.

The deflecting surface 62 is provided by a supporting body 64 which is connected to the main body 34 by means of elongate connecting elements 66. There are four connecting elements 66 in the illustration. In further variants, more or fewer connecting elements 66 may be present.

The connecting elements 66 may be releasably connected to the main body 34 and/or the supporting body 64. This allows to use different supporting bodies 64 (and components associated with and/or contained in it as described below), in order to e.g. provide deflecting surfaces of different shapes. This also permits to use the arrangement without supporting bodies.

In addition to areas that provide the deflecting surface 62, the supporting body 64 comprises areas underneath the deflecting surface 62 in which a swirling device 68 is at least partly arranged. The swirling device 68 comprises a drive 70, e.g. an electric motor, and an element 72 operatively connected to it which may be driven by the drive.

The drive 70 may be arranged in a space 74 in the area of the supporting body 64 that provides the deflecting surface 62, as shown. In further variants, the drive 70 may be arranged further below in the illustration, in particular in areas of the supporting body 64 underneath the area of the supporting body 64 providing the deflecting surface 62.

The operative connection between the drive 70 and the element 72 may be achieved by means of a drive shaft 76.

The element 72 is arranged in a space 78 which is in fluid communication with the interior of the aerosol wetting chamber 4. Openings 80 are provided for this purpose in the illustration.

In further variants, the element 72 may be disposed freely at a bottom side of the supporting body 64. This may e.g. be achieved by a drive shaft coupled to the drive 70, which extends from the supporting body 64 and has the element 72 attached at its free end.

The element 72 may be formed as a single vane, wing or rotor blade or comprise several vanes, wings or rotor blades. In the latter cases, the element 72 may be compared to the rotor of a fan for illustration purposes.

By moving the element 72, a swirl is created in the aerosol wetting chamber 4 which supports the distribution of aerosol. In particular, the element 72 is formed and/or is moved such that a swirl is created which is substantially horizontal in the illustration. Such a swirl may ensure that aerosol can also be transported to the peripheral regions of the aerosol wetting chamber 4. Such a swirl may ensure that aerosol can also be transported to the peripheral regions of the aerosol wetting chamber 4, creating a homogeneous aerosol concentration in the aerosol wetting chamber. Accordingly, a thoroughly mixed test atmosphere may be created in the chamber.

The connecting members 66, as shown, may be hollow, tubular. The interior space of a connecting member 66 may be used to feed one or several lines 82 via which the drive 70 may be supplied with energy and/or may be controlled. It is provided in such variants that a corresponding port 84 is provided on the main body 34, e.g. as a socket. A line feed-through 86 may extend through the main body 34 from the port 84 to the corresponding connecting member 66.

In further variants, two tubular connecting members 66 are arranged such that the respective interior space is in fluid communication with the fluid supply passageway or fluid discharge passageway, respectively, to supply or discharge fluid to be supplied to or discharged from the aerosol wetting chamber 4 via the connecting members 66.

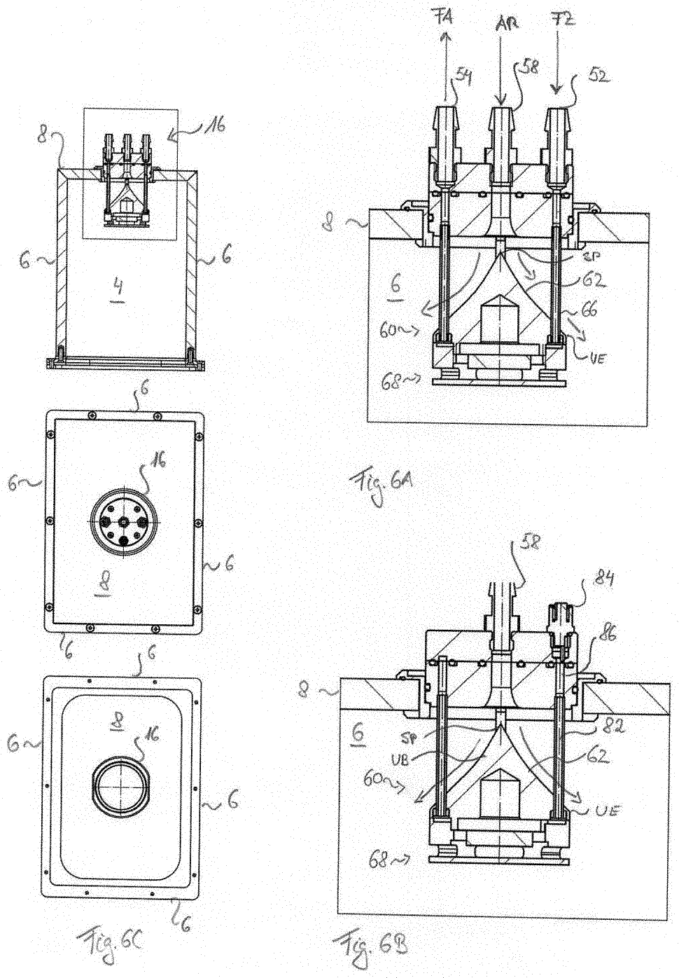

The variant of FIG. 6A-6C may be considered as variant of the variant of FIG. 5A-5D, in which the aerosol receiving device 16 is replaced by another type of aerosol supply to the aerosol passageway 36 of the main body 34. Furthermore, the ports for fluid supply and discharge and the port for the swirling device may be arranged differently. Apart from such differences, the comments above regarding the previous drawings shall apply, unless otherwise stated.

In the variant of FIG. 6A-6C, the main body 34 also comprises an aerosol passageway 36. The aerosol passageway 36 here can be formed differently, in particular with a smaller diameter, as it is not necessary to connect the connecting member of an aerosol receiving device, or to arrange it there.

The passageway 36 of FIG. 6A to 6C includes an inlet 46 and an outlet 38. The inlet 46 here is in fluid communication with an aerosol port 58 to which an aerosol feed line can be connected in order to supply aerosol of an external source.

The main body 34 in this case does not need to be configured to correspond to aerosol receiving device, but can be formed independently thereof. Thus, the main body 34 can e.g. be flatter, as illustrated in FIG. 6A-6C.

A flat build may result in there not being enough installation space on the sides of the main body 34, in order to arrange the ports for fluid supply and discharge and/or the port for the swirling device.

As shown in FIG. 6A-6C, it is provided in such cases to arrange the ports on the upper side of the main body 34.

List of reference numerals

| | |
|---|---|
| 2 | Aerosol wetting arrangement |
| 4 | Aerosol wetting chamber |
| 6 | Side walls |
| 8 | Top |
| 10 | Base |
| TV | Temperature regulator |
| 12 | Hood |
| 14 | Sample well |
| 16 | Aerosolization device |
| 18 | Pipetting |
| 20 | Aerosol receiving device |
| 22 | Housing of the aerosol receiving device |
| 24 | Aerosol generating chamber of the aerosol receiving |
| | device/aerosolization device |
| 26 | Inlet of the aerosol receiving device/aerosolization device |
| 28 | Outlet of the aerosol receiving device/aerosolization device |
| 30 | Membrane of the aerosol receiving device/aerosolization device |
| 32 | Connecting part of the aerosol receiving device/aerosolization |
| | device |
| 34 | Main body |
| 36 | Aerosol passageway |
| 38 | Outlet of the aerosol passageway |
| AR | Discharge direction |
| 40 | Receiving opening of a hood of an aerosol wetting chamber |
| 42 | Step/recess of the housing |
| 44 | Sealing element of the housing |
| 46 | Inlet of the aerosol passageway |
| 48 | Fluid supply passageway |
| 50 | Fluid discharge passageway |
| 52 | Port for fluid supply passageway |
| 54 | Underside of the main body |
| 56 | Port for fluid discharge passageway |
| FZ | Fluid supply direction |
| FA | Fluid discharge direction |
| 58 | Aerosol port |
| 60 | Aerosol deflector |
| 62 | Deflecting surface |
| SP | Upper end (tip) of the deflecting surface |
| UB | Transition area of the deflecting surface |
| UE | Lower end of the deflecting surface |
| 64 | Supporting body |
| 66 | Connecting element for connecting the supporting body and the |
| | main body |
| 68 | Swirling device |
| 70 | Drive of the swirling device |
| 72 | Movable element (rotor blade) |
| 74 | Space for the drive of the swirling device |
| 76 | Drive shaft |
| 78 | Space for movable element (rotor blade) |
| 80 | Openings for fluid communication |
| | Space for movable element (rotor blade) |
| 82 | Line(s) |
| 84 | Port for the drive |
| 86 | Line feed-through |

What is claimed is:

1. An aerosolization device for an aerosol wetting chamber comprising a receiving opening for receiving the aerosolization device and adapted for wetting samples disposed in the aerosol wetting chamber with aerosol, the aerosolization device comprising:
a main body with an aerosol passageway, which includes an outlet for discharging aerosol in a discharge direction (AR),
an aerosol deflector disposed downstream of the outlet in the discharge direction (AR) and having at least one deflecting surface extending transverse to the discharge direction (AR) and adapted to deflect aerosol emerging from the outlet in a direction different from the discharge direction, (AR),
a swirling device arranged downstream of the aerosol deflector in the discharge direction (AR) and comprising at least one movable element adapted to set surrounding fluid in motion.

2. The aerosolization of claim 1, wherein
the at least one deflecting surface comprises at least three corners, or
there are at least two deflecting surfaces, each extending transverse to the discharge direction (AR), wherein an angle between the discharge direction (AR) and the respective plane of the deflecting surface is the same or different, or
the at least one deflecting surface has the shape of a lateral surface of a cone with a first angle between the cone axis and the surface line of the cone, which is adjoined by the lateral surface of a truncated cone with a second angle between the cone axis and the surface line of the truncated cone, or
the at least one deflecting surface has the shape of a lateral surface
of a cone or truncated cone, or
of a sphere or hemisphere, or
of a segment of a sphere or hemisphere, or
of a cylinder or segment of a cylinder, or
of a prism or prism segment, or
of a pyramid or truncated pyramid.

3. The aerosolization device of claim 1, wherein the movable element of the swirling device=comprises at least one vane (wing, rotor blade) that is rotatable about a rotation axis.

4. The aerosolization device of claim 1, wherein the movable element of the swirling device is arranged in a space that comprises at least one opening.

5. The aerosolization device of claim 1, wherein the at least one deflecting surface is formed at a supporting body and the supporting body comprises the space in which the movable element of the swirling device is arranged.

6. The aerosolization device of claim 1, wherein the supporting body comprises a seat for at least partially receiving the swirling device.

7. The aerosolization device of claim 1, wherein the main body comprises at least one fluid supply passageway comprising an inlet for supplying fluid to be supplied to the aerosol wetting chamber, and an outlet for discharging the fluid to be supplied to the aerosol wetting chamber into the aerosol wetting chamber; and/or
the main body comprises at least one fluid discharge passageway comprising an inlet for supplying fluid to be discharged from the aerosol wetting chamber, and an outlet for discharging fluid to be discharged from the aerosol wetting chamber into the aerosol wetting chamber.

8. The aerosolization device of claim 1, wherein the main body comprises at least one line feed-through adapted to feed through a line for controlling and/or supplying energy to the swirling device.

9. The aerosolization device of claim 1, further comprising: a port for an aerosol feed line for feeding aerosol of an external source, the port being arranged upstream of the aerosol passageway and being in fluid communication with the aerosol passageway.

10. The aerosolization device of claim 1, further comprising an aerosol receiving device with an aerosol generation chamber comprising an inlet for loading a substance for producing an aerosol into the aerosol generating chamber, and an outlet for feeding aerosol produced from the substance from the aerosol generating chamber to the aerosol passageway.
